(19) United States
(12) Patent Application Publication
(10) Pub. No.: US 2024/0067793 A1
(43) Pub. Date: Feb. 29, 2024

(54) ENZYMATIC RECYCLING OF POLYURETHANES BY A COMBINATION OF CUTINASES AND LIPASES

(71) Applicant: SOCIETE DES PRODUITS NESTLE S.A., Vevey (CH)

(72) Inventors: TIM BORNER, Epalinges (CH); VANESSA SREBNY, Singen (DE); NINA CHRISTINA MARIA ROOTHANS, TX Delft (NL)

(21) Appl. No.: 18/259,159

(22) PCT Filed: Dec. 13, 2021
(86) PCT No.: PCT/EP2021/085373

§ 371 Date: Jun. 23, 2023

(30) Foreign Application Priority Data
Dec. 24, 2020 (EP) 20217179.9

(51) Int. Cl. C08J 11/10; C12N 9/20; C12R 1/845

(52) U.S. Cl. CPC C08J 11/105; C12N 9/20; C08J 2375/04; C12R 2001/845; C12Y 301/01074

(57) ABSTRACT

The present invention relates generally to the field of degrading polyurethane (PU), for example PU layers in multi-layer packaging. For example, the present invention relates to a method of degrading polyurethane (PU) comprising the step of subjecting the PU to an enzyme cocktail comprising at least one lipase and at least one cutinase. The PU may be a PU-based layer in a multilayer packaging structure comprised in a packaging. Remarkably, the subject matter of the present invention allows the efficient selective degradation of PU containing layers in multi-layer packaging materials.

### FIELD OF THE INVENTION

The present invention relates generally to the field of degrading polyurethane (PU), for example PU layers such as adhesives and coatings typically used in multi-layer food packaging applications. For example, the present invention relates to a method of degrading polyurethane (PU) in packaging material comprising the step of subjecting the packaging material comprising the PU to at least one cutinase in combination with at least one lipase. The PU may be a PU-based layer in a multilayer packaging structure comprised in a packaging. Remarkably, the subject matter of the present invention allows the efficient selective degradation of PU containing layers in multi-layer packaging materials.

### BACKGROUND OF THE INVENTION

Plastic production has been increasing for over the last six decades, reaching 348 million tonnes in 2017 (Plastics Europe, 2018). Packaging is the major sector of plastic usage, with almost 40% of the market demand (Plastics Europe, 2018). It consists for a large part of single-use plastics, which have a short lifetime, turning to waste shortly after being acquired by the consumer. It is common knowledge that plastic accumulation is a current major environmental concern, resulting from the high resistance of plastics to degradation, together with improper disposal or deposition of waste in landfills. Yet, efforts have been made over the past years to avoid plastic deposition in landfills (Plastics Europe, 2018). Nevertheless, a large amount of packaging plastics still ends up as waste, so efficient recycling technologies are needed to simultaneously minimize the amount of produced waste and the resource consumption to produce plastics.

Polymers used in packaging can be divided into two main groups: the ones with a carbon-carbon backbone [e.g., polypropylene (PP), polyethylene (PE), polyvinyl chloride (PVC) and polystyrene (PS)] and those with a heteroatomic backbone [e.g., polyesters and polyurethanes (PU)]. The high energy required to break C—C bonds makes hydrocarbons very resistant to degradation (Microb Biotechnol, 10(6), 1308-1322). On the other hand, polyesters and polyurethanes have hydrolysable polyester bonds so they are less resilient to abiotic and biotic degradation.

The most common polyester is polyethylene terephthalate (PET) (Plastics Europe, 2018). Plastic packaging is usually not composed of one single polymer. Instead, blends or multiple layers of different polymers are often required to obtain certain properties (elasticity, hydrophilicity, durability or water and gas barrier) related to the specific application of the plastic (Process Biochemistry, 59, 58-64). Also, packaging materials generally contain adhesives, coatings and additives, such as plasticizers, stabilizers and colorants (Philos Trans R Soc Lond B Biol Sci, 364(1526), 2115-2126). This makes the recycling of some packaging materials very difficult.

Current plastic waste recycling technologies predominantly consist of thermo-mechanical processes, while chemical recycling is in its early industrialization phase. Mechanical recycling requires clean input waste streams that may be achieved through prior cleaning and separation steps in the case of contaminated and complex packaging structures, respectively. Thus, the recycling rates of multilayer packaging today are very low. Instead, multilayer packaging is mostly incinerated or ends up in landfills. Besides, the mechanical recycling process often results in downgraded plastics with decreased properties and limited food grade quality, thus losing their original value and application. These materials are then typically used for lower-value secondary products. On the other hand, chemical recycling processes are being developed to enable the recovery of the polymer's building blocks that can be used to remake the plastic. However, this process is economical and energetically costly and usually requires extreme conditions and harsh chemicals. These technologies are thus not ideal for complex, multilayer plastic materials (Process Biochemistry (2017), 59, 58-64).

A technology enabling the selective removal and recycling of each component of multilayer plastic packaging would provide the possibility of reproducing the original packaging and expanding recycling to mixed plastic packaging waste and materials.

Enzymes are very selective towards their substrate, so they offer a high potential to be applied in recycling processes. Enzymes would enable the selective decomposition of each layer into either the starting building blocks, which can be used for subsequent production of new plastics or as added-value chemicals. The enzymatic and microbial degradation of recalcitrant plastics has been increasingly studied over the past years, with particular focus on PET (Microb Biotechnol, 10(6), 1302-1307). Even though the enzymatic degradation of plastic is difficult, there are enzymes capable of degrading polyesters used in the production of plastic packaging. The degradation efficiency of enzymes however varies with different classes and types of enzymes, and the conditions under which the experiments were carried out highly influence the extent of degradation. In addition, the polymer properties, e.g., crystallinity and composition, also have a strong influence on the rate of degradation.

Even though efforts have been made to increase the efficiency of enzymatic degradation of polymers, most studies were performed on pure materials. Although these studies provide a good initial insight on the enzymatic degradation of plastics, they are not representative of actual packaging materials as polymers are not isolated in this case and additives may be present. Moreover, a deep understanding of the effect of experimental conditions, enzyme properties and polymer properties on the degradation process is lacking.

Therefore, to design a selective recycling process for multi-layer packaging is of high importance.

It would therefore be desirable to have available a process that can be used to selectively degrade PU-based layers, such as adhesives and coatings in multi-layer packaging that is cost efficient, results in high quality materials and does not require harsh processing conditions.

Any reference to prior art documents in this specification is not to be considered an admission that such prior art is widely known or forms part of the common general knowledge in the field.

### SUMMARY OF THE INVENTION

The objective of the present invention was, hence, to enrich or improve the state of the art and in particular to provide the art with a method to efficiently degrade polyurethane used in packaging material, for example a polyurethane layer in a multi-layer packaging that does not require prior separation of layers, does not require harsh chemicals and/or harsh conditions, and offers economic and environmental advantages, or to at least provide a useful alternative to solutions available in the art.

The inventors were surprised to see that the objective of the present invention could be achieved by the subject matter of the independent claim. The dependent claims further develop the idea of the present invention.

Accordingly, the present invention provides a method of degrading polyurethane (PU) comprising the step of subjecting the PU to an enzyme cocktail comprising at least one lipase and at least one cutinase.

As used in this specification, the words “comprises”, “comprising”, and similar words, are not to be interpreted in an exclusive or exhaustive sense. In other words, they are intended to mean “including, but not limited to”.

The present inventors were surprised to find that cutinases and lipases act synergistically in the degradation of PU. PU, for example, is typically used in food packaging applications as adhesives or coatings. The inventors have obtained particularly promising results with the cutinase Thc_Cut2 in combination with a lipase, for example RoL. Remarkably, enzyme cocktails comprising at least one lipase and at least one cutinase could be used to selectively and efficiently degrade PU typically used in as glue or coating in multilayer packaging. For example in the case of a PE based multilayer packaging structure that comprises a PU-based layer, it was possible by using enzyme cocktails comprising at least one lipase and at least one cutinase to selectively degrade the PU-based layer, so that the PU monomers could be recovered, and the PE-based backbone of the multilayer packaging structure could be liberated and subjected to PE recycling. The clean state of the resulting PE allowed that the recycled PE could be recycled for further applications.

### BRIEF DESCRIPTION OF THE DRAWINGS

Certain advantages and embodiments of the present invention are apparent from the attached examples, tables and figures. The figures show the following:

FIGS. 1A and 1B depict the enzymatic degradation of commercial coating Adcote 545-75 using enzyme combinations of RoL and Thc_Cut2. The reactions were carried out on 0.79 mg Adcote 545-75 in 0.2 ml t 37° C. and enzyme loadings of 25.6 μg/mg polymer for the single enzymes and each enzyme in combination (in total 51.2 μg/mg polymer). (A) Total polymer release profile of enzymatic hydrolysis with RoL (Δ, empty triangle), Thc_Cut_2 (○, empty circle) and Thc_Cut2+RoL (♦, filled diamond). For a negative control, only buffer was added (●, filled circle), while the positive control contained 1M NaOH (▪ dashed). (B) Depicts the degradation gain of the combination (white bars) compared to the single enzyme activities (grey and black bars respectively) after a reaction time of 24 h. A negative control was performed each time with polyurethane solely in 0.1 M PBS buffer at pH 7. Each bar represents the average percentage of polymer mass released from duplicate reactions.

FIGS. 2A and 2B demonstrate the enzymatic degradation of Adcote 17-35 using enzyme combinations of RoL and Thc_Cut2. The reactions were carried out on 0.79 mg Adcote 17-35 in 0.2 ml t 37° C. and enzyme loadings of 25.6 μg/mg polymer for the single enzymes and each enzyme in combination (in total 51.2 μg/mg polymer). (A) Total polymer release profile of enzymatic hydrolysis with RoL (Δ, empty triangle), Thc_Cut_2 (○, empty circle) and Thc_Cut2+RoL (♦ filled diamond). For a negative control, only buffer was added to vials (●, filled circle), while the positive control contained 1M NaOH (▪ dashed). (B) Depicts the degradation gain of the combination (white bars) compared to the single enzyme activities (grey and black bars respectively) after a reaction time after 24 h. A negative control was performed each time with polyurethane solely in 0.1 M PBS buffer at pH 7. Each bar represents the average percentage of polymer mass released from duplicate reactions.

### DETAILED DESCRIPTION OF THE INVENTION

Consequently, the present invention relates in part to a method of degrading polyurethane (PU) comprising the step of subjecting the PU to an enzyme cocktail comprising at least one lipase and at least one cutinase.

The PU may be provided as pure material or as a material comprising PU.

The inventors have obtained, for example, very good results, when the material comprising PU was a polyester-containing polyurethane-based polymer. For example, the inventors have obtained excellent results with coatings and adhesives that are polyurethane-based with aliphatic and aromatic polyester segments.

In accordance with the present invention the PU is degraded by an enzyme cocktail comprising at least one lipase and at least one cutinase. The term “degradation” comprises de-polymerization, which refers to the process of converting a polymer into smaller polymer chains, oligomers and eventually monomers. The term “degradation” more generally describes that the polymer chain is cleaved by at least one of the enzymes, resulting in shorter polymer chains and/or release of monomers. Such polymer fragmentation can for example be achieved through the activity of endo-acting enzymes or through the incomplete activity of exo-acting enzymes. In one embodiment of the present invention the method of the present invention may be a method of de-polymerizing PU, for example at least one PU-based layer in a packaging.

Cutinases catalyze the hydrolytic reaction of cutine and water to yield cutine monomers. Cutinases belong to the family of serine esterases, usually containing the Ser-His-Asp triad of serine hydrolases.

The at least one cutinase may be a cutinase from a fungal or microbial source. Using enzymes from a fungal or a microbial source have the advantage that they can be naturally produced, and—in particular, if the enzymes are enzymes that are secreted by the fungus or the micro-organism—the fungus or the micro-organism itself can be used to degrade the at least one polymer layer in a packaging material.

The at least one cutinase may be a cutinase from Thermobifida fusca, Thermobifida cellulosilytica.

Thermobifida organism is a thermophilic bacteria occurring in soil and is a major degrader of plant cell walls in heated organic materials such as compost heaps, rotting hay, manure piles or mushroom growth medium. Its extracellular enzymes have been studied because of their thermostability, broad pH range and high activity.

The inventors have obtained particularly promising results, when the at least one cutinase was Thc_Cut2, while other cutinases selected from the group consisting of Thf_Cut1, Thc_Cut1 showed improvements as well. These cutinases produced even better synergistic results than other cutinases when used in a cocktail with lipases.

Thf_Cut1 (T. fusca), Thc_Cut1 (T. cellulosilytica), Thc_Cut2 (T. cellulosilytica) as well as the metagenomic cutinase BC-CUT-013 were purchased from Biocatalyst Ltd. UK and were recombinantly produced in E. coli.

Lipases are enzymes that catalyzes the hydrolysis of lipids. The inventors have obtained particularly promising results, when the at least one lipase was a lipase from Rhizopus oryzae. The inventors have obtained a very good synergistic effect with cutinases, when the lipase was RoL. RoL is a lipase from Rhizopus oryzae and was purchased from Sigma-Aldrich (Switzerland).

The enzymes may be used in pure form. However, the inventors were surprised to see that the enzymes could also be used as crude extracts, for example, as crude extract from a fungal and/or microbial source. Using a crude extract has the advantage that an expensive purification of the enzymes is not necessary. Consequently, in accordance with the present invention the at least one lipase and/or the at least one cutinase may be used as a crude extract. Advantageously, the at least one lipase and/or the at least one cutinase may be used as a water soluble, crude extract.

The amount of enzyme used is not critical for the success of the degradation step in the method of the present invention. It is, however, important for the speed of the degradation. The inventors have obtained good results when the degradation was carried out with an enzyme loading of at least about 0.5 μg protein/mg polymer, at least about 5 μg protein/mg polymer, or at least about 50 μg protein/mg polymer.

The inventors recommend adjusting the ratio of cutinase and lipase to achieve an optimal synergy. The precise optimal ratio will depend on the specific enzymes used, but in general the inventors recommend to use the at least one cutinase and the at least one lipase in a unit ratio in the range of about 10:1 to 1:10, for example of about 5:1 to 1:5, further for example of about 2:1 to 1:2. In their experiments the inventors have obtained very good results when the unit ratio of the at least one cutinase and the at least one lipase was about 1:1.

In particular if the cutinase and/or the lipase used in the framework of the present invention is obtainable from a thermophilic organism, the cutinase and/or the lipase will also exhibit a certain thermo-stability. Accordingly, the degradation can be carried out at elevated temperatures, for example at a temperature in the range of 30-40° C., 35-45° C. or 40-50° C. The degradation at elevated temperatures will proceed significantly faster. The expected increase in reaction speed can be estimated in accordance with the Arrhenius equation.

However, elevating the reaction temperature will cause costs, for example for the increase in energy usage. Hence, it may be preferred if the degradation is carried out at ambient temperature. This is, in particular, the case if the required reaction time is not critical. Ambient temperature may differ depending, for example, on geographic location and on the season. Ambient temperature may mean for example a temperature in the range of about 0-30° C., for example about 5-25° C.

Accordingly, for example, in the framework of the present invention, the PU may be subjected to the enzyme cocktail comprising at least one lipase and at least one cutinase at a temperature in the range of 20-50° C., for example 30-40° C. The inventors have obtained very good results at a temperature of about 37° C.

The inventors have further tested the reaction at different pH values. It was found that the method of the present invention was most effective, if the degradation was carried out at neutral to slightly alkaline conditions. Good results were obtained at a pH in the range of 6-9. For example, the PU may be subjected to the enzyme cocktail comprising at least one lipase and at least one cutinase at a pH in the range of about 6-9, for example in the range of about 6.5-8.

Accordingly, it may be preferred if the degradation is carried out at pH in the range of about 7-9, preferably in the range of about 7.5-8.5, for example at a pH of about 8.2.

The inventors have obtained good results when the PU was subjected to the enzyme cocktail comprising at least one lipase and at least one cutinase for at least 24 hours, 3 days, for at least 10 days, or for at least 20 days.

With the method of the present invention a partial or even a complete degradation of the PU appears possible. The inventors conclude this from a corresponding release of reporter molecules. For example, it appears possible with the method of the present invention to degrade the PU by at least 10 weight-%, at least weight-%, at least 20 weight-%, at least 25 weight-%, at least 30 weight-%, at least 35 weight-%, at least 45 weight-%, at least 50 weight-%, or at least 55 weight-%. This degradation resulted in part in the generation of monomers or monomer mixtures. Accordingly, in the method of the present invention the degradation of the at least one polymeric layer results in the generation of at least 10 weight-%, at least 15 weight-%, at least 20 weight-%, at least 25 weight-%, at least 30 weight-%, at least 35 weight-%, at least 45 weight-%, at least 50 weight-%, or at least 55 weight-% of the monomers or monomer mixtures of the degraded polymer.

The method of the present invention is—in particular—well suited for application in packaging recycling. Accordingly, in the framework of the present invention, the PU may be present in a packaging, for example in food packaging or pet food packaging. For the purpose of the present invention, the term “food” shall be understood in accordance with Codex Alimentarius as any substance, whether processed, semi-processed or raw, which is intended for human consumption, and includes drink, chewing gum and any substance which has been used in the manufacture, preparation or treatment of “food” but does not include cosmetics or tobacco or substances used only as drugs.

Multilayer packaging structures are frequently used in the industry today, for example in the food industry. Here, multi-layered packaging is often used for light weight packaging to provide certain barrier properties, strength and storage stability to food items. Such a multi-layered packaging material may be produced by lamination, or coextrusion, for example. Further, techniques based on nanotechnology, UV-treatments and plasma treatments are used to improve the performance of multi-layer packaging. Compr Rev Food Sci Food Saf. 2020; 19:1156-1186 reviews recent advances in multilayer packaging for food applications.

If the packaging comprises a multi-layer packaging material, the multi-layer packaging material may comprise at least two polymeric layers.

The polymeric layers may comprise a PU-based layer and at least one layer selected from the group consisting of a further PU-based layer, a polyethylene terephthalate (PET)-based layer, a polyethylene (PE)-based layer, or a combination thereof. The PU-based layer may be a PU-based adhesive or a PU-based coating.

A layer shall be considered PU, PE or PET based, if it contains at least about 50 weight-%, at least about 60 weight-%, at least about 70 weight-%, at least about 80 weight-%, at least about 90 weight-%, at least about 95 weight-%, or at least about 99 weight-% of PU, PE or PET, respectively.

PU layers are frequently used in food packaging. PU layers are typically flexible films with high elongation, inherently strong, flexible, and free of plasticizers, that do not become brittle with time. They are resistant to fat and hydrolysis within a large range of temperatures typically experienced for packaging during manufacturing and usage. They can withstand elevated temperatures and exhibit excellent resistance to microbiological attacks.

PET layers are also frequently used in food packaging. They are typically transparent, have a very good dimensional stability and tensile strength and are stable over wide temperature ranges. PET layers show low water adsorption behavior, are significantly UV-resistant and provide a good gas barrier. Furthermore, it is easy to print on PET in high quality. The moisture barrier properties of PET films are, however, only moderate. Today's mechanical recycling technologies for PET yield lowered recyclate quality and limited food grade application.

Polyethylene (PE) is a plastic polymer that is relatively easy to recycle mechanically, nowadays. PE thermoplastics interestingly become liquid at their melting point and do not start to degrade under elevated temperatures. Hence, such thermoplastics can be heated to their melting point, cooled, and reheated again without significant degradation. Upon liquification of PE due to heat, PEs can be extruded or injection molded and—consequently—recycled and used for a new purpose. However, it is problematic to recycle PEs if—e.g., in a multi-layer packaging material—a PE layer is combined with other plastic layers.

One advantage of the method described in the present invention is that it can be used to delaminate selectively PU layers from a PE layer. Consequently, the method of the present invention may be used for the selective delamination of at least one PU-based layer in a multilayer packaging.

The inventors could show that the enzyme cocktail used in the framework of the present invention can degrade PU-based layers. For example, the inventors have shown that commercially available polyurethanes could be degraded with the cutinases and lipases used in the framework of the present invention.

In the method of the present invention, the PU may be present in a packaging comprising a multilayer packaging structure, wherein the multilayer packaging structure comprises a base layer that can be recycled, for example a PE-based layer, and at least one PU-based layer, wherein the method is used to recycle the multilayer packaging structure by degrading the at least one PU-based layer and by subjecting the base layer to a recycling stream. The resulting PU monomers can be collected and reused as well.

Many multilayer packaging structures comprise a PE-based layer, a PET-based layer and a PU-based layer. The inventors have shown that an enzyme cocktail comprising at least one lipase and at least one cutinase can be used to degrade PU-based layers. The use of cutinases to biodegrade PET is known, for example, from Nature Scientific Reports (2019) 9:16038. Consequently, in one embodiment the present invention relates to a method of degrading multilayer packaging structures comprising at least one PU-based layer and at least one PET based layer comprising the step of subjecting the multilayer packaging structure to an enzyme cocktail comprising at least one lipase and at least one cutinase.

In a further embodiment of the method of the present invention, the packaging comprises a multilayer packaging structure comprising at least three polymeric layers, wherein the polymeric layers comprises at least one PU-based layer, at least one PET based layer and at least one PE-based layer wherein the method comprises the step of subjecting the multilayer packaging structure to an enzyme cocktail comprising at least one lipase and at least one cutinase, and subjecting the PE-based layer to further recycling. The generated building blocks of the PU-based layer and/or the PET-based layer may be collected for reuse.

In scope of the presented invention, the inventors also propose its application for multilayer packaging that are comprised of more than three polymeric layers. For example, polyvinyl alcohols (PVHOs), such as EVOH and BVOH used for oxygen barrier, are typically found in addition to PU-, PET- and PE-layers and would be released from the multilayer besides PE when subjected to at least one cutinase and at least one lipase as described in this invention.

For example, in the food industry, many packings comprise 4-5 layers. One typical example is a multilayer packaging material with the structure PET/PU/EVOH/PU/PE. In one embodiment of the present invention, the method of the present invention may be used to degrade a multilayer packaging material comprising or consisting of the structure PET/PU/EVOH/PU/PE. Optionally, such a packaging material may be metallized, for example aluminized, for example with an AlOx coating.

The inventors further propose that the degradation speed and/or completeness can be significantly increased, if the surface to volume ratio of the packaging, for example the multilayer packaging structure is increased. For example, the packaging may be mechanically treated to reduce the particle size to particles with an average diameter of less than about 5 mm, less than about 1 mm, or less than about 0.5 mm diameter before subjecting the packaging to the enzyme cocktail. Typically, the mechanical treatment may be shredding, for example. Hence, the method of the present invention may further comprise the step of reducing the particle size of the PU and/or the PU containing material, for example the PU containing packaging, before or during subjecting the PU and/or the PU containing material to an enzyme cocktail comprising at least one lipase and at least one cutinase. The particle size may be reduced by a mechanical treatment to particles with an average diameter of less than about 5 mm, less than about 1 mm, or less than about 0.5 mm diameter.

One advantage of the method of the present invention is that it can be carried out under controlled conditions, for example in a closed vessel, such as a bioreactor, for example. The relatively gently conditions of the degradation process do not require bioreactors that can withstand extreme conditions, which in turn contributes to the cost effectiveness of the method of the present invention. Using a closed vessel in turn has the advantage that reaction and process parameters, such as temperature and agitation, for example, can be precisely controlled.

Those skilled in the art will understand that they can freely combine all features of the present invention disclosed herein. In particular, features described for the method of the present invention may be combined. Further, features described for different embodiments of the present invention may be combined.

Although the invention has been described by way of example, it should be appreciated that variations and modifications may be made without departing from the scope of the invention as defined in the claims.

Furthermore, where known equivalents exist to specific features, such equivalents are incorporated as if specifically referred in this specification. Further advantages and features of the present invention are apparent from the figures and non-limiting examples.

### EXAMPLE 1: ENZYME COCKTAIL OF LIPASES AND CUTINASES DEGRADING COMMERCIAL POLYESTER-BASED PUS (ADCOTE 102A, ADCOTE 545-75 AND ADCOTE 17-3)

### Materials and Methods

### Materials and Chemicals

The polyurethane materials Adcote 102A (36% w/w), Adcote 545-75 (75% w/w), Adcote 17-3 (75% w/w) and co-reactant F (75% w/w) were thankfully provided by Dow Chemicals. Glycerol, K₂HPO₄, KH₂PO₄, fluorescein, fluorescein dilaurate, sodium hydroxide (NaOH) and ethyl acetate were all purchased from Sigma.

Based on analysis of degradation products by liquid chromatography high resolution mass spectrometry (LC-HRMS) following monomer contents could be confirmed (see Table 1). All materials contain phthalic acid as well as diethylene glycol. Adcote 102 A and Acote 17-3 also contain both sebacic acid as diacid component, whereas Adcote 545-75 contains adipic acid. For the coating neo-pentyl-di-propanol could be detected. Co-reactant F was described in patents to contain isocyanate terminated polyol based branched pre-polymers. The isocyanate component was found to be toluene di-isocyanate (Wu et al, 2019, US20190284456A1).

TABLE 1
The table below lists the three materials tested (Adcote 102A, Adcote 545-75
and Adcote 17-3) their preparation and identified components by LC-MS.

| Name | Adcote 102A | | Adcote 545-75 | Adcote 17-3 | Co-Reactant F |
|---|---|---|---|---|---|
| Description | Polyester component of two | | Polyester component | solvent-based coating | Cross-linked, |
| | component based adhesive | | of two component | | isocyanate |
| | | | based adhesive | | containing |
| | | | | | component |
| Preparation | cured with co-reactant F | | Cured with co-reactant | Already cured | Cured with |
| | | | F | | polyester |
| | | | | | component |
| Identified Components | 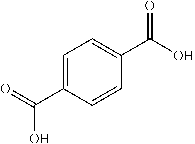 [SMILES: O=C(O)c1ccc(C(=O)O)cc1] | 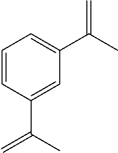 | 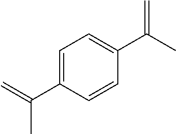 | 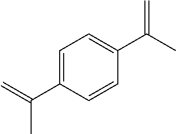 | 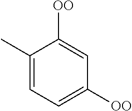 |
| | 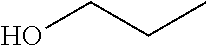 | 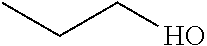 | 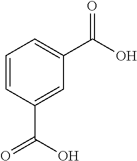 [SMILES: O=C(O)c1cccc(C(=O)O)c1] | 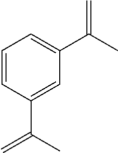 | 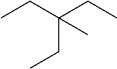 |

| | | | | | | |
|---|---|---|---|---|---|---|
| 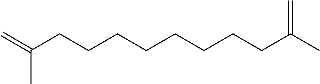 | 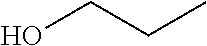 | 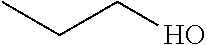 | 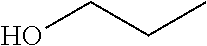 | 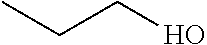 | 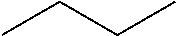 | 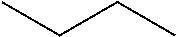 |

| | |
|---|---|
| 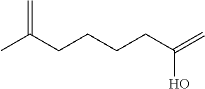 | 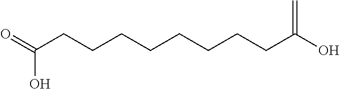 |
| | 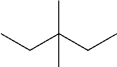 |

Thf_Cut1 (T. fusca), Thc_Cut2 (T. cellulosilytica) and Thc_Cut1 (T. cellulosilytica) as well as the metagenomic cutinase BC-CUT-013 were purchased from Biocatalyst Ltd. UK. All of these enzymes used were used as crude extract, non-purified, which represents a more industrially relevant and cheaper preparation than purified enzymes that are too costly for such proposed waste application. The lipases RoL (Rhizopus oryzae lipase) and PcL (Pseudomonas cepacia lipase) were both purchased in purified form from Sigma-Aldrich (Switzerland).

TABLE 2
List of enzymes investigated, their type, abbreviation, organism
of origin, production organism, quality and supplier.

| | | | | Production | | |
|---|---|---|---|---|---|---|
| Name | Abbreviation | Type | Organism | Organism | Quality | Supplier/Produce [symbol] |
| T. fusca | Thf_Cut | Cutinase | T. Fusca | Recombinant | Crude | Biocatalysts Ltd. |
| cutinase | | | | E. coli | soluble | UK |
| T. cellulosilytica | Thc_Cut2 | Cutinase | T. | | extract | |
| cutinase 2 | | | cellulosilytica | | | |
| T. cellulosilytica | Thc_Cut1 | Cutinase | T. | | | |
| 1 | | | cellulosilytica | | | |
| Biocatalyst | BC-CUT-013 | Cutinase | Metagenomic | | | |
| Cutinase 013 | | | | | | |
| Rhizopus | RoL | Lipase | Rhizopus | | Purified | Sigma 62305-1G- [symbol] |
| oryzae lipase | | | oryzae = | | | |
| | | | Rhizopus | | | |
| | | | arrhizus | | | |
| | | | | | Purified | Sigma 62305-25G [symbol] |
| Pseudomonas | PcL | Lipase | Pseudomonas | | Purified | Sigma 62309- |
| cepacia lipase | | | cepacia | | | 100MG |

[symbol]

indicates data missing or illegible when filed

All enzymes were diluted to stock solutions of 1 mg/ml protein in 40% (w/v) glycerol for easier handling during experiments except for Pseudomonas cepacia Lipase which was diluted to 0.1 mg protein/ml due to higher purity.

The degree of adhesive and coating degradation by enzymes was measured via the following methods: fluorescent release assay for indirect estimation of polymer degradation and LC-MS identification of specific degradation products proofing hydrolysis into polymer building blocks (oligomers and monomers).

Upon receiving of the materials, 2.5× (polyester component) and 5× (co-reactant) stock solutions (w/w) were prepared by diluting the polymer in ethyl acetate. For the adhesives Adcote 102A and Adcote 545-75 the co-reactant had to be mixed with the polymer in ratios of 4.5:100 (w/w) and 11.5:100 (w/w) respectively.

### Preparation of Polyurethane Coated 96 Well Plates

The indirect fluorescent assay, established by Zumstein and colleagues (Zumstein, M. T., et al. (2017) Environmental Science & Technology 51(13): 7476-7485) is based on the assumption that the release of a homogeneous embedment reporter molecule (fluorescein dilaurate, FDL) in the target polymer matrix (adhesive or coating) is directly correlated to the degree of degradation of same polymer material. Only upon material degradation, FDL is released out of the polymer matrix and can then be hydrolysed by an esterase-active enzyme into laureate and fluorescein, of which the latter molecule can be quantified fluorometrically (51.21/494 nm). One percent (%) polymer degradation is defined as one % release of originally embedded reporter molecule, in this case corresponding to 0.1 wt % incorporated FDL which was the optimum amount to reach a high detection limit while minimizing the effect on the polymer matrix and enzymes.

The stock solutions were used to prepare the casting solutions in ethyl acetate containing 2.3% (w/w) polymer and 0.0023% (w/w) FDL. This corresponds to a FDL:polymer ratio of 1:1000.

For 0.79 mg polymer per well, 40 μl of the casting solution were transferred to solvent-resistant 96-well plates (Greiner 6551.209, Greiner Bio-One), before leaving them to cure for 1 week at room temperature.

Enzyme activity screening of 15 enzyme combinations against Adcote 102A, 545-75 and 17-3

The reaction was carried out in 200 μl 0.1 M PBS (potassium-saline phosphate)Buffer adjusted to pH 7 using an enzyme loading of 25.6 μg protein/mg polymer for a single enzyme reaction and an enzyme ratio (mg/mg protein) of 1:1 for the enzyme combinations (in total 51.2 μg protein/mg polymer). The buffer was chosen to ensure pH stability as acids are formed upon hydrolysis that may affect the enzyme negatively. The buffer was prepared by mixing K₂ HPO₄ and KH₂PO₄ according to the Henderson-Hasselbalch equation.

As a positive control reaction for the assay, the FDL-loaded polymer sample was exposed to a 1M sodium hydroxide (NaOH) solution as stability of long-chain fluorescein diesters and greatly decreases above pH 8.5 (Guilbault, G. G., & Kramer, D.

N. (1966), 14(1), 28-40). In addition, ester bonds as in polyesters as well as urethanes bonds present in polyester-polyurethanes can be hydrolysed at elevated pH as reported in a study by Matuszak and colleagues (Matuszak, M. L., Frisch, K. C., & Reegen, S. L. (1973), Journal of Polymer Science: Polymer Chemistry Edition, 11(7), 1683-1690). Thus, a basic solution of 1M NaOH is used as positive control for the indirect FDL assay.

As a negative control, FDL-loaded polymer sample were exposed to the respective buffer solution without enzyme or NaOH. Leakage of FDL was determined negligible. All plates were incubated at 37° C. at 250 rpm and measured after 0, 2, 4, 6, 8, 10 and 24 h at 494/521 nm in a plate reader.

A fluorescein calibration curve of 0.03125-5 μM was used to calculate the FDL release. After 24 h the reaction the reaction was stopped and all plates stored at −20° C.

### Results and Discussion

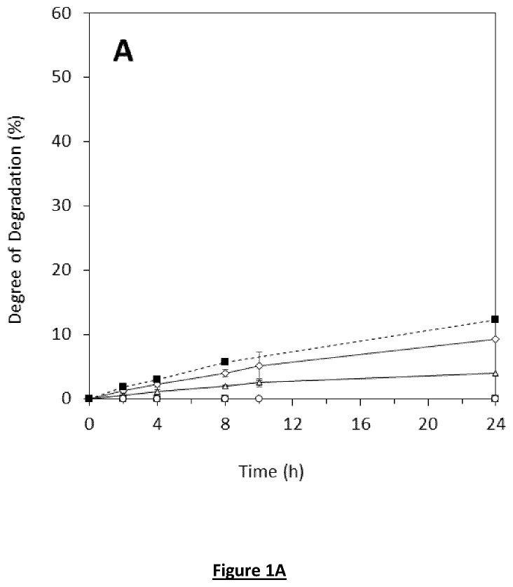

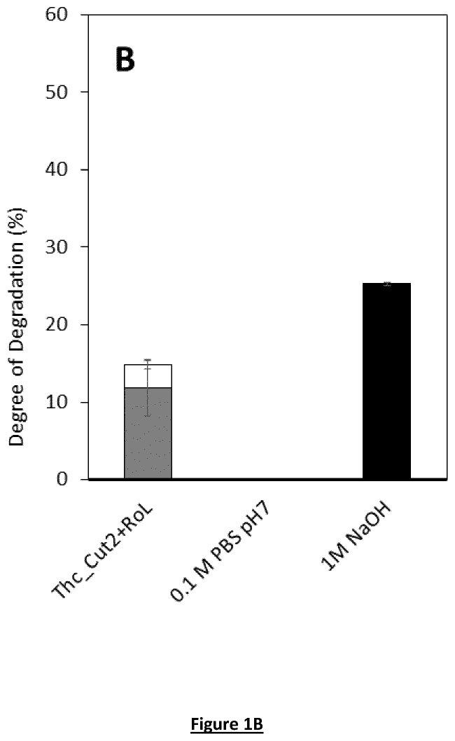

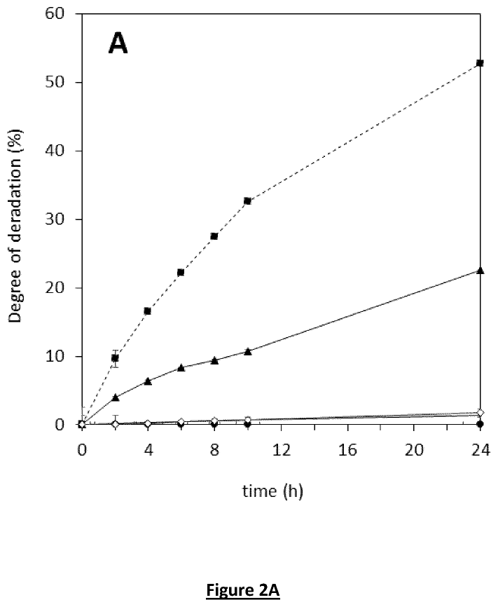

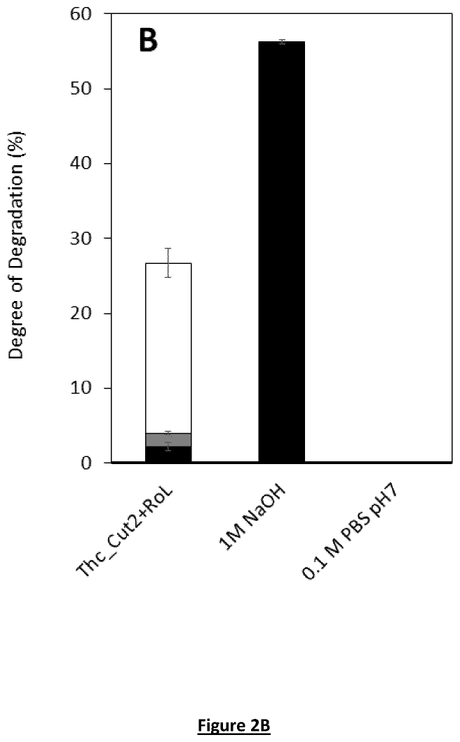

The inventors discovered that the combination of a lipase with a cutinase enhances the degradation efficiency of PU materials. FIGS. 1 and 2 shows the most effective enzyme combinations for the commercial PU-based adhesives and coating. As shown in FIG. 2a-b, the degradation efficiency was enhanced from 2.2% to 26.7% when combining RoL and BC-CUT-013 in a ratio of 1:1 (mg/mg) on the PU coating Adcote 17-3. The enzymatic degradation of the adhesive Adcote 545-75 was also enhanced by using the same enzyme cocktail (FIG. 1a-b) though with a less strong effect as for Adcote 17-3.

The combination of different enzyme types has been reported to provide an increase in the degradation efficiency of complex substrates like cellulose (e.g., cellulases and monooxygenases), but only few studies on polyesters (Barth, M. et al. 2015. Biochemical Engineering Journal, 93, 222-228) and polyurethanes. Polyurethanes have been subjected to enzyme cocktails by combining different types of enzyme, such as, esterase and amidase (Magnin, A., et al. 2019. Waste Management, 85, 141-150) or esterases and a protease (Ozsagiroglu, et al. 2012. Polish Journal of Environmental Studies 21.6: 1777-1782), of which however, former could only detect the release of minor building blocks but no higher mass release and latter only found a competitive (negative) effect. Hence, this invention provides a new, effective enzyme combination for PU-based coating that drastically enhances the degradation of PU-based polymers.

The inventors hypothesize that the drastic degradation gain by combining a cutinase (Thc-Cut2) and a lipase (RoL) in this invention is based on a synergistic effect, for example, of complementary substrate specificity that allows the elimination of inhibitory degradation products by one enzyme to enhance the activity of the other, or the enzyme combination introduces a complementary endo- and an exo-activity, or complementary cleavage sites that enable a more broad hydrolysis at various polymer locations thereby leading to a faster and more comprehensive PU degradation.

The enzyme combination Thc_Cut2 and RoL demonstrated superior activity compared to using only the single enzymes for Adcote 545-75 and Adcote 17-3. The inventors point out that the degradation gain of 12-fold as shown in FIG. 2b compares to the sum of degradation of the two individual enzymes with the degree of degradation of enzyme cocktail and thus depicts the real synergistic gain of the enzyme combination. Notably, combinations of other enzymes, for example, were found to exhibit no or even negative effects and thus the single degradation activity was the same or higher than in combination (data not shown).

The application of enzyme combination could be used in a more efficient and faster decoating (Adcote 17-3) and delamination (Adcote 545-75) process, respectively, of multi-layered materials.

For the recycling of laminates and polyurethane coated packaging, the selective degradation of the polyurethane layer is the key to separate layers and enabling their subsequent individual recycling. Furthermore, most of enzymatic degradation studies of polyurethanes studies have been carried out on custom-made PU polymers and not on commercial industrially relevant PU polymers and formulations. This may be due to the much more complex and diverse chemical composition, especially in proprietary formulations, which complicates the analysis of enzymatic degradation process. Here, the inventors could demonstrate a 12× fold increase of degradation based on the indirect release of FDL for the best enzyme combination Thc_Cut2 and RoL at ambient temperatures of 37° C. for the coating Adcote 17-3 (see FIG. 2). Similarly, the degree of degradation increased 1.26 fold for this combination on Adcote 545-75 (see FIG. 1b).

The combination could be used in a more efficient and faster decoating or delamination process of multilayered materials, requiring much less of the single enzyme components, thereby reducing economic costs and environmental impact.

1. Method of degrading polyurethane (PU) comprising the step of subjecting the PU to an enzyme cocktail comprising at least one lipase and at least one cutinase.

2. Method in accordance with claim 1, wherein the at least one cutinase is Thc_Cut2; and the at least one lipase is RoL.

3. Method in accordance with claim 1 wherein the at least one cutinase and the at least one lipase is used as a crude extract.

4. Method in accordance with claim 1 wherein the at least one cutinase and the at least one lipase is used with an enzyme loading of at least 12 μg protein per mg polymer.

5. Method in accordance with claim 1 wherein the at least one cutinase and the at least one lipase are used in a unit ratio in the range of about 10:1 to 1:10.

6. Method in accordance with claim 1 wherein the PU is subjected to the enzyme cocktail at a temperature in the range of 20-50° C.

7. Method in accordance with claim 1 wherein the PU is subjected to the enzyme cocktail at a pH in the range of about 6-9.

8. Method in accordance with claim 1 wherein the PU is subjected to the enzyme cocktail for at least 3 days.

9. Method in accordance with claim 1, wherein the PU is present in a packaging, wherein the packaging comprises a multilayer packaging structure comprising at least two polymeric layers, wherein the polymeric layers comprises a PU-based layer and at least one layer selected from the group consisting of a further PU-based layer, a polyethylene terephthalate (PET)-based layer, a polyethylene (PE)-based layer, and a combination thereof.

10. Method in accordance with claim 9, wherein the PU is present in the packaging in a PU-based adhesive or a PU-based coating.

11. Method in accordance with claim 1, wherein the method is used for the selected delamination of at least one PU-based layer in a multilayer packaging.

12. Method in accordance with claim 1, wherein the PU is present in a packaging comprising a multilayer packaging structure, wherein the multilayer packaging structure comprises a base layer that can be recycled, for example a PE-based layer, and at least one PU-based layer, wherein the method is used to recycle the multilayer packaging materials by degrading the at least one PU-based layer and by subjecting the base layer to a recycling stream.

13. Method in accordance with claim 1, wherein the method further comprises the step of reducing the particle size of the PU.

14. Method in accordance with claim 13, wherein the particle size is reduced by a mechanical treatment to particles with an average diameter of less than about 5 mm.

15. Method in accordance with claim 1, wherein the method is carried out in a closed vessel.
